(19) United States
(12) Patent Application Publication
(10) Pub. No.: US 2023/0050187 A1
(43) Pub. Date: Feb. 16, 2023

(54) Methods for Treating Parkinson's Disease

(71) Applicant: MeiraGTx UK II Limited, London (GB)

(72) Inventors: Alexandria Forbes, New York, NY (US); Matthew During, Rowayton, CT (US)

(21) Appl. No.: 17/784,510

(22) PCT Filed: Dec. 14, 2020
(86) PCT No.: PCT/US2020/064928

§ 371 Date: Jun. 10, 2022

Related U.S. Application Data
(60) Provisional application No. 62/947,418, filed on Dec. 12, 2019.

(51) Int. Cl. A61K 38/51; A61K 48/00; A61P 25/16

(52) U.S. Cl. CPC A61K 38/51; A61K 48/0008; A61P 25/16

(57) ABSTRACT

Disclosed are methods for treating neurological disorders such as Parkinson's disease (PD) using glutamic acid decarboxylase (GAD) and identifying PD patients that will be most receptive to the method of treating PD. In one aspect, the disclosure provides a method of treating PD in a subject in need thereof, the method comprising: (a) identifying a subject having less than about 10 hours, and preferably less than about 8 hours, of on-time per day; and (b) administering to the subject a composition comprising a therapeutically effective amount of one or more vectors to the subthalamic nucleus of the patient, wherein each vector comprises a nucleic acid sequence encoding glutamic acid decarboxylase (GAD) and wherein the subject's on-time is increased.

### CROSS REFERENCE TO RELATED APPLICATION

This application claims priority to U.S. Provisional Application No. 62/947,418 filed on Dec. 12, 2019, which is incorporated by reference herein in its entirety.

### FIELD OF THE INVENTION

The present disclosure relates to methods and compositions for treating Parkinson's disease (PD) and other neurodegenerative disorders. More particularly, methods comprising administering to a subject one or more vectors comprising nucleic acid sequences encoding an isoform of glutamic acid decarboxylase (GAD) and identifying target patient populations that will be most receptive to the method of treating PD are described herein.

### BACKGROUND

Central nervous system disorders, particularly those disorders that involve dopamine neurotransmitters, affect millions of people around the world every year. More than 10 million people worldwide and nearly one million patients in the U.S. are living with Parkinson's disease (PD), one of the most common central nervous system disorders.

PD is a multifactorial disease involving both genetic and non-genetic factors. Some mechanisms that may contribute to the development of PD include the accumulation of misfolded proteins aggregates, failure of protein clearance pathways, mitochondrial damage, oxidative stress, excitotoxicity, neuroinflammation, and genetic mutations. PD affects the nerve cells parts of the brain called the basal ganglia and the substantia nigra. The substantia nigra is a basal ganglia structure located in the midbrain that plays an important role in reward and movement. This area is predominantly composed of dopaminergic neurons (DA), which produce the neurotransmitter dopamine. In the brain, dopamine functions as an inhibitory neurotransmitter that regulates the excitability of neurons, which are involved in controlling balance and body movement. In normal brains, DA neurons release dopamine which crosses the synapse and fit into receptors on the receiving cell. That cell is stimulated to pass the message on. After the message is passed on, the receptors release the dopamine molecules back into the synapse, where the excess dopamine is “taken up” or recycled within the releasing neuron.

Gamma-Aminobutyric acid, or γ-aminobutyric acid (GABA) is the chief inhibitory neurotransmitter in the developmentally mature mammalian central nervous system. Most of the nigra1 DA neurons express both Gamma-aminobutyric acid (A) ((GABA (A)) and Gamma-aminobutyric acid (B) (GABA (B)) receptors. The DA neurons modulate a monosynaptic GABA output in the substantia nigra. The recycling of dopamine is modulated by Gamma-aminobutyric acid (GABA) pathway. It is well known in the art that activation of GABA pathway causes an increase in dopamine and in turn reduce the rate of firing of nerve cells. The firing pattern of dopaminergic neurons is also effectively modulated by GABAergic inputs in vivo. Unavailability of GABA (A) receptors causes dopaminergic neurons to shift to a burst firing pattern regardless of the original firing pattern increasing spontaneous firing rate.

For reasons not yet fully understood, the dopamine-producing nerve cells of the substantia nigra begin to die off in PD patients, which causes a deficit of dopamine and a loss of signaling through dopamine receptors in polysynaptic neurons. Additionally, the scarcity of DA neurons causes reduction in the gamma-aminobutyric acid (GABA) inhibitory input to the subthalamic nucleus, leading to increased activity in the subthalamic nucleus. The subthalamic nucleus in turn sends signal for increased activity to other cells within the basal ganglia. When GABA levels fall below a certain threshold, it causes dopamine depletion in the brain. The loss of dopamine alters the activity of neurons within the basal ganglia, causing uncontrolled firing of nerve cells. When dopamine levels drop below a certain point (about 80% decrease), the symptoms of PD such as uncontrolled muscle movements, tremor begins to occur.

PD is characterized by a progressive deterioration in the muscle movements of the body; poor balance and coordination; and uncontrolled trembling. The standard treatment of PD involves oral administration of the dopamine precursor L-3,4-dihydroxyphenylalanine (levodopa or L-Dopa), which eliminates symptoms associated with PD, but does not ultimately prevent the degeneration of dopaminergic cells. Thus, currently used treatments for PD merely reduce PD symptoms without slowing or halting disease progression.

Administration of L-Dopa allows a PD patient to have “on-time”, a period of time in which a PD patient has adequate control of PD symptoms. When the effect of L-Dopa wears off, the symptoms of PD reemerge. This time period is referred to as “off-time”. The measurement of on-time and off-time are typically calculated by asking the patients to keep a medication diary. In this diary, the patients record how long it takes for L-Dopa to kick in and to wear off. In the early stages of PD, a patient's on-time is about 16 hours with the administration of L-Dopa. However, as the disease progresses, the amount of on-time gradually decreases even with larger doses of medication. Additionally, the side effects associated with long-term administration of L-dopa can be quite severe, and include mental changes such as depression, hallucination, mania, delusions, agitations, and excessive sleeping. Administration of L-dopa can also have a detrimental effect in patients with cardiovascular or pulmonary, renal, hepatic or endocrinal diseases. Some of the side effects associated with long-term administration of L-dopa can be mitigated by co-administration of N-amino-α-methyl-3-hydroxy-L-tyrosine monohydrate, an inhibitor of aromatic amino acid decarboxylase (AADC), an enzyme that decarboxylates L-Dopa to dopamine. However, this drug combination still may cause nausea, dyskinesia, psychosis, and hypotension.

One of the chief barriers of treating PD with small molecule drugs is that most systematically administered drugs are not able to cross the blood brain barrier. One approach has focused on increasing the lipid content of polypeptides to facilitate their transport across the blood brain barrier. Another approach has concentrated on enhancing the permeability of capillaries in the brain. However, none of these approaches has solved the problem of crossing blood-brain barrier. Other approaches of treating PD, such as transplanting engineered cells that produce dopamine to the brain and deep brain stimulations, are characterized by severe side effects, such as high mortality rate, an increased chance of severe infection, and potential brain damage. More importantly, none of the available drugs address the underlying cause of PD or provide PD patients more on-time without serious side effects.

In view of the current limitations in the treatment of PD, there remains an unmet need for (1) a non-transient treatment of PD, particularly in PD patients who are not responding to the current standard of care, (2) the non-transient treatment that effectively prolongs on-time in PD patients without causing significant side effects, and (3) a reliable method for identifying patient populations who will be most receptive to the non-transient treatment of PD.

### SUMMARY OF THE INVENTION

In one aspect, the disclosure provides a method of treating PD in a subject in need thereof, the method comprising: (a) identifying a subject having less than about 10 hours, and preferably less than about 8 hours, of on-time per day; and (b) administering to the subject a composition comprising a therapeutically effective amount of one or more vectors to the subthalamic nucleus of the patient, wherein each vector comprises a nucleic acid sequence encoding glutamic acid decarboxylase (GAD) and wherein the subject's on-time is increased. In one embodiment, the one or more vectors are introduced bilaterally to the subthalamic nucleus of the patient.

In one embodiment, the disclosure provides a method of treating PD in a subject in need thereof, where the subjects has less than 10 hours, less than 9 hours, less than 8 hours, less than 7 hours, less than 6 hours, less than 5 hours, or less than 4 hours of on-time per day before treatment.

In one embodiment, the disclosure provides a method of treating PD in a subject in need thereof, wherein the one or more vectors comprises a nucleic acid sequence encoding GAD-65 and/or a nucleic acid sequence encoding GAD-67. In embodiments, two vectors are administered, wherein one vector comprises a nucleic acid encoding GAD-65 and the other vector comprises a nucleic acid encoding GAD-67. In further embodiments, the vector comprising a nucleic acid encoding GAD-65 and the vector comprising a nucleic acid encoding GAD-67 are administered in a ratio of about 1:2 to about 2:1, preferably about 1:1.

In one embodiment, the disclosure provides a method of treating PD in a subject in need thereof, wherein the one or more vectors used in the method comprises a nucleic acid sequence encoding GAD-65 and encoding GAD-67. In one embodiment, the amino acid sequence of human GAD-65 is provided as SEQ ID NO: 1 (Genbank Accession No. NM000818; M81882). In another embodiment, the amino acid sequence of human GAD-65 is provided as SEQ ID NO: 3. In some embodiments, the nucleic acid sequence encodes a protein that is at least 80%, at least 85%, at least 90%, at least 95%, at least 96%, at least 97%, at least 98%, or at least 99% identical to the sequence of SEQ ID NO: 1 or of SEQ ID NO: 3. In embodiments, the nucleic acid sequence encoding GAD-65 comprises SEQ ID NO: 2 or SEQ ID NO: 4. In some embodiments, the nucleic acid sequence encoding GAD-65 comprises a sequence that is at least 90%, at least 95%, at least 96%, at least 97%, at least 98%, or at least 99% identical to the sequence of SEQ ID NO: 2 or of SEQ ID NO: 4. In one embodiment, the amino acid sequence of human GAD-67 is provided as SEQ ID NO:5 (Accession No. M81883). In another embodiment, the amino acid sequence of human GAD-67 is provided as SEQ ID NO: 7. In some embodiments, the nucleic acid sequence encodes a protein that is at least 80%, at least 85%, at least 90%, at least 95%, at least 96%, at least 97%, at least 98%, or at least 99% identical to the sequence of SEQ ID NO: 5 or SEQ ID NO: 7. In embodiments, the nucleic acid sequence encoding GAD-67 comprises SEQ ID NO: 6. In embodiments, the nucleic acid sequence encoding GAD-67 comprises SEQ ID NO: 8. In embodiments, the nucleic acid sequence encoding GAD-67 comprises a sequence that is at least 90%, at least 95%, at least 96%, at least 97%, at least 98%, or at least 99% identical to the sequence of SEQ ID NO:6 or SEQ ID NO: 8.

In one embodiment, the disclosure provides a method of treating PD in a subject in need thereof, where one or more vectors used in the method are viral vectors. In one embodiment, the disclosure provides a method of treating PD in a subject in need thereof, where the viral vectors disclosed are adeno-associated virus (AAV) vectors.

In one embodiment, the disclosure provides a method of treating PD in a subject in need thereof, where the composition comprises at least 1×10¹¹ vector genomes/ml. In one embodiment, the disclosure provides a method of treating PD in a subject in need thereof, where the composition comprises at least 3×10¹¹ vector genome/ml. In one embodiment, the disclosure provides a method of treating PD in a subject in need thereof, where the composition comprises at least 1×10¹² vector genome/ml.

In one embodiment, the disclosure provides a method of treating PD in a subject in need thereof, wherein the subject shows an average increase of at least 20%, at least 30%, at least 40% in on-time 12 months post treatment compared to pre-treatment. In embodiments, the subject has an increase in on-time of about 1, 1.5, 2, 2.5, 3, 3.5, 4, 4.5, or 5 hours 12 months following treatment.

### BRIEF DESCRIPTION OF THE FIGURES

FIG. 1. Increase in “ON” Time Following AAV-GAD Gene Therapy. Subjects receiving either AAV-GAD (GAD) into the STN or sham surgery (sham) recorded hourly ON and OFF times in a diary throughout each day for two weeks at each of the listed time points (1, 3, 6 and 12 months after surgery). The average daily ON and OFF time over the two week period determined from the diaries were compared with baseline diaries taken within the 30 days prior to surgery in order to determine the change in the number of hours of ON time following surgery. There was an overall difference between groups across the entire 12 month study period by analysis of variance (ANOVA; p=0.44). Two-tailed t-tests showed significant increases in ON time at 3 and 12 months following surgery (p<0.05) and a strong trend toward significance at 1 month. The average increase in ON time was between 1 and 2.5 hours across the study in the AAV-GAD group, with minimal to no average change in the sham group at any time point.

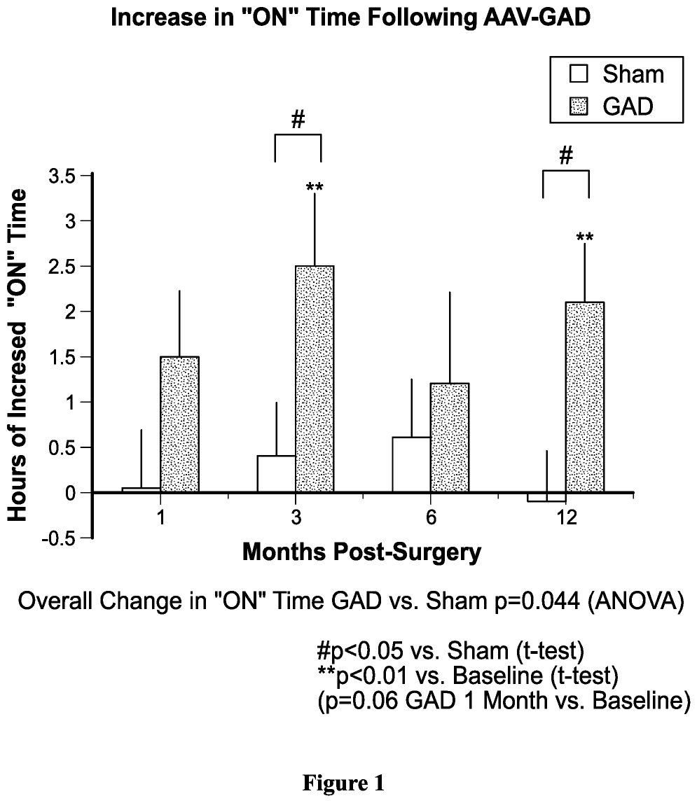

FIG. 2. Correlation Between Baseline ON Time and Change in ON Time Following AAV-GAD Gene Therapy. At 12 months following surgery, the change in average daily ON time from baseline per subject in the AAV-GAD treatment group was correlated against the change in clinical scores for the same subjects across the same time period. The clinical score used was part 3 of the Unified Parkinson's Disease Rating Scale (UPDRS), which is the gold-standard rating of motor function for Parkinson's Disease patients. This shows a strong correlation between baseline ON time and increase in ON time at 12 months following subthalamic AAV-GAD gene therapy (r=−0.59) indicating that fewer hours of ON time at baseline (more severe patients) predicted a greater increase in the number of hours of ON time (greater response) following treatment. Subjects with lower baseline on-time showed increased gain of on-time post treatment. Patients with less than 8 hours of ON time have the greatest response to AAV-GAD therapy, with an average of 3.5 hour per day increase in ON time.

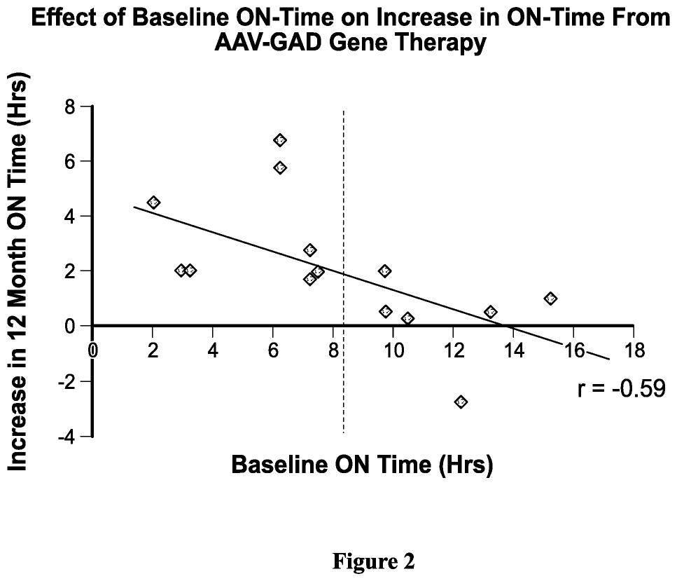

### DETAILED DESCRIPTION OF THE INVENTION

The present disclosure provides methods for treating PD in a subject in need thereof, as well as methods of identifying PD patients that will be most receptive to the method of treating PD. In one aspect, provided is a method of treating PD by identifying a subject having less than 10 hours, less than 9 hours, less than 8 hours, less than 7 hours, less than 6 hours, less than 5 hours, less than 4 hours of on-time per day and administering a therapeutically effective amount of GAD (e.g., by expressing GAD-65 and GAD-67) in the subthalamic nucleus of the patient to increase the on-time.

### Patient Selection

In one aspect, the disclosure provides a method of treating patients who can most benefit from the treatment provided in the disclosure. In some embodiments, the patient has an on-time of about 10 hours or less per day. In some embodiments, the patient has about 9 hours or less, about 8 hours or less, about 7 hours or less, about 6 hours or less, or about 5 hours or less of on-time per day. At the beginning of the disease, typically within first 3-5 years of diagnosis, a single dose of L-Dopa allows the patients to have an on-time of about 16 hours. As the disease progresses, the period of on-time becomes progressively shorter even with increasing doses of L-Dopa medication. The intermediate stage of PD, typically about 5-10 years after diagnosis, 50-70% of patients suffer L-dopa induced motor complications. At this stage, the L-Dopa benefit wears off after about 4 hours or less, and patients begin to fluctuate between on-time and off-time motor responses. With increasing disease progression, the duration of response after a single dose of L-Dopa becomes progressively shorter and may ultimately mirror the plasma half-life of L-Dopa (approximately 60 to 90 minutes) for the advanced stage PD patients (typically >10 years after diagnosis). In some cases, patients may fail to respond to a given dose of L-Dopa. The most difficult clinical situation is described as the true “on-off” or yo-yo phenomenon, in which patients rapidly fluctuate between on-time and off-time states with a seeming loss of relationship to the L-Dopa dose. Dyskinesia and off-time periods are severe at this stage, and it may be difficult or impossible to satisfactorily control patients with available therapy. Additionally, the intermediate and advanced stage patients suffer adverse side effects due to increased L-Dopa medication.

The standard treatment of patients with stage 3 or higher PD is a combination of L-dopa and an AADC inhibitor. This therapy causes the PD symptoms to temporarily subside, thus allowing the patient to regain control of his or her faculties. As used herein, the period of time during which a PD patient experiences relief from PD symptoms is referred to as “on-time.” PD symptoms relieved during on-time include, but are not limited to, loss of motor control, pain, slurring, tremor, and impaired balance. As the effect of medication wears off, the tremors and other PD symptoms return. As used herein, the period of time in which patients experience a reemergence of PD symptoms due to wearing off of medication, is referred to as “off-time.”

The measurement of on-time and off-time can be determined by medication diary kept by the patients. Patients note down the time when they feel the medication took effect, allowing them to have control on their motor facilities and the time when they feel that the medication is wearing off, causing the PD symptoms to reemerge. Clinicians use the timings noted in the diary to determine the on-time and off-time for a PD patient at a particular point of time. On-time and off-time may also be measured using wearable devices including but not limited to motion sensors, accelerometers and posture detection devices by, e.g., measuring and tracking hypokinetic and hyperkinetic features associated with PD.

In embodiments, the patient has had PD for at least three years before receiving the therapy described herein. In some embodiments, the patient has PD for at least four, or at least five years. In embodiments, the patients receiving the therapy described herein are in in stage 2, stage 3, stage 4, or stage 5 of the disease. The progression of PD can be divided into five stages. Stage 1 of PD is characterized by disturbances in the facial expression, speech and/or locomotion. The symptoms are initially only seen on one side of the body (unilateral involvement), and there is usually minimal or no functional impairment. During stage 2 of PD, both hemispheres of the brain become affected by the disease. As a result, tremors gradually become bilateral and can affect the patient's midline. Additional symptoms of PD in stage 2 may include the loss of facial expression on both sides of the face, decreased blinking, speech abnormalities, soft voice, monotone voice, slurring speech, stiffness or rigidity of the muscles in the trunk that may result in neck or back pain, stooped posture, and general slowness in activities of daily living. Stage 3 is characterized by loss of balance and slowness of movement. Balance is compromised by the inability to make the rapid, automatic, and involuntary adjustments necessary to prevent falling, and falls are common at this stage. A stage 4 patient shows severe and limiting symptoms. The patient may be able to stand without assistance, but movement may require a walker. Stage 5 is the most advanced and debilitating stage of PD. Stiffness in the legs may make it impossible to stand or walk. The patient requires a wheelchair or is bedridden. Around-the-clock nursing care is required for all activities for patients at this stage of PD.

In embodiments, the patient, or patients, selected for the therapy disclosed herein have a score of about 20 or more, about 30 or more, or about 40 or more on part III of the UPDRS in the medication state and/or have motor complications caused by administration of L-Dopa. The stages of PD can be measured using the Unified Parkinson's Disease Rating Scale (UPDRS) (see, e.g., Metman et al, Mov. Disord. 19:1079-1084 (2004)). The UPDRS scale includes a series of ratings for typical PD symptoms. The scale is composed of four parts: part I assesses behavioral problems such as intellectual decline, hallucinations, and depression; part II assesses patients' perceptions of their ability to carry out activities of daily living, including dressing, walking, and eating; part III covers the evaluation of motor disability and includes ratings for tremor, slowness (bradykinesia), stiffness (rigidity), and balance; and part IV covers a number of treatment complications including ratings of involuntary movements (dyskinesias), painful cramps (dystonia), and irregular medication responses (motor fluctuations). Part III or the motor examination is scored through a structured neurological examination by a clinician. It consists of 14 items that can have scores from zero (normal) to four (severe). The scores are added to give an overall score of the involuntary movements. Clinicians use the score obtained from the evaluation of the items listed under part III to determine the severity of PD and appropriate treatment.

### Methods of Treating PD

In embodiments, the present disclosure provides methods of treating, preventing, and/or reducing the severity or extent of PD, by administering to a subject in need thereof a therapeutically effective amount of a composition, or compositions, comprising one or more vectors comprising a nucleic acid encoding GAD-65 and/or vectors comprising a nucleic acid encoding GAD-67. In embodiments, the method for treating PD patients comprises administering one or more compositions to a patient in need thereof wherein the one or more compositions comprise a vector, or vectors, for the expression of a GAD-65 and/or a vector for the expression of a GAD-67.

As used herein, “treating”, “treat”, “treatment” refer to slowing down, relieving, ameliorating, or alleviating at least one of the symptoms of the disease or disorder, or reversing one or more symptoms of the disease or disorder after its onset. The object of the treatment is to prevent or lessen or halt an undesired physical condition, disorder or disease or to obtain beneficial clinical results.

The terms “prevent”, “prevention”, and the like refer to acting prior to overt disease or disorder onset, to prevent the disease or disorder from developing or to minimize the extent of the disease or disorder, or slow its course of development.

The term “cure” and the like means to heal, to make well, or to restore to good health or to allow a time without recurrence of disease so that the risk of recurrence is small.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

In embodiments, the subject receiving the therapy described herein (e.g., receiving a therapeutically effective amount of a composition comprising one or more vectors comprising a nucleic acid encoding GAD-65 and/or vectors comprising a nucleic acid encoding GAD-67) has an average increase in on-time of about 1, about 1.5, about 2, about 2.5, about 3, about 3.5, or about 4 hours at 12 months after receiving the therapy. In embodiments, the subject has about a 15%, about a 20%, about a 25%, about a 30%, about a 35%, about a 40%, about a 45%, or about a 50% increase in on-time at 12 months after receiving the therapy.

PD standard treatment involves administration of a dopamine precursor, L-Dopa, to replenish the dopamine. However, increasing GABA levels also causes these uncontrolled movements to cease as the higher concentration of GABA allows the dopamine level to return or increase. In embodiments, the disclosure provides a method of increasing GABA levels in the subthalamic nucleus, by administering one or more vectors comprising glutamic acid decarboxylase (GAD) isoforms. GABA is produced in the brain by GAD which catalyzes the decarboxylation of glutamate to GABA and CO₂. The mammalian brain expresses two different isoforms of GAD, GAD-65 and GAD-67, named for their respective molecular weights of 65 and 67 kDa. These isoforms in combination provide a dual system for controlling neuronal GABA concentration. In humans, the genes for GAD-67 and GAD-65 are located on different chromosomes (GAD1 and GAD2 genes are located on chromosomes 4 and 10, respectively.). GAD-65 and GAD-67 show significant differences in their levels of expression in different brain regions. GAD-67 is expressed uniformly throughout the brain whereas GAD-65 expression is concentrated primarily in the axon terminals. Together, these two enzymes maintain most of the physiological supply of GABA in mammals. Human GAD-65 cDNA encodes a polypeptide of 585 amino acid residues (Genbank Accession No. NM000818; M81882). In an embodiment, the amino acid sequence of human GAD-65 is provided as SEQ ID NO: 1. In another embodiment, the amino acid sequence of human GAD-65 is provided as SEQ ID NO: 3. Human GAD-67 encodes a polypeptide of 594 amino acid residues (Genbank Accession No. M81883). In an embodiment, the amino acid sequence of human GAD-67 is provided as SEQ ID NO:5 (Genbank Accession No. M81883). In another embodiment, the amino acid sequence of human GAD-67 is provided as SEQ ID NO: 7.

In embodiments, the vector comprises a nucleic acid sequence encoding a GAD isoform. In one embodiment, the nucleic acid sequence encodes GAD-65. In one embodiment, the nucleic acid sequence encodes GAD-67. In embodiments, the vector comprises a nucleic acid sequence encoding GAD-65 and a nucleic acid sequence encoding GAD-67. In embodiments, the nucleic acid sequence encoding GAD-65 comprises a sequence encoding a protein of SEQ ID NO: 1. In other embodiments, the nucleic acid sequence encoding GAD-65 comprises a sequence encoding a protein of SEQ ID NO: 3. In some embodiments, the nucleic acid sequence encodes a protein that is at least 80%, at least 85%, at least 90%, at least 95%, at least 96%, at least 97%, at least 98%, or at least 99% identical to the sequence of SEQ ID NO: 1 or of SEQ ID NO: 3. In one embodiment, the nucleic acid sequence encoding GAD-65 comprises SEQ ID NO: 2 or SEQ ID NO: 4. In some embodiments, the nucleic acid sequence encoding GAD-65 comprises a sequence that is at least 90%, at least 95%, at least 96%, at least 97%, at least 98%, or at least 99% identical to the sequence of SEQ ID NO: 2 or of SEQ ID NO: 4.

In embodiments, the nucleic acid sequence encoding GAD-67 comprises a sequence encoding a protein of SEQ ID NO: 5 or of SEQ ID NO: 7. In some embodiments, the nucleic acid sequence encodes a protein that is at least 80%, at least 85%, at least 90%, at least 95%, at least 96%, at least 97%, at least 98%, or at least 99% identical to the sequence of SEQ ID NO:5 or of SEQ ID NO: 7. In embodiments, the nucleic acid sequence encoding GAD-67 comprises SEQ ID NO: 6. In embodiments, the nucleic acid sequence encoding GAD-67 comprises SEQ ID NO: 8. In embodiments, the nucleic acid sequence encoding GAD-67 comprises a sequence that is at least 90%, at least 95%, at least 96%, at least 97%, at least 98%, or at least 99% identical to the sequence of SEQ ID NO:6 or SEQ ID NO: 8.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package, BLASTP, BLASTN, FASTA, and the ALIGN program (version 2.0). The well-known Smith Waterman algorithm may also be used to determine similarity. The BLAST program is publicly available from NCBI and other sources. In comparing sequences, these methods account for various substitutions, deletions, and other modifications.

In some embodiments, the sequence encoding GAD-65 and/or the sequence encoding GAD-67 are codon-optimized.

The disclosure provides methods of treating, preventing, and/or reducing the severity or extent of PD symptoms by administering to a subject in need thereof a therapeutically effective amount of a composition comprising a first vector comprising a nucleic acid encoding GAD-65 and a second vector comprising a nucleic acid encoding GAD-67. In embodiments, the ratio for the two vectors (one encoding GAD-65 and the other encoding GAD-67) in the composition is from about 2:1 to about 1:2. For example, when the vector is an AAV vector, the ratio of viral particles comprising nucleic acid encoding GAD-65 to viral particles comprising nucleic acid encoding GAD-67 is from about 2:1 to about 1:2, and in particular about 1:1.

In some embodiments, the method comprises administering a therapeutically effective amount of a first composition comprising a vector comprising a nucleic acid encoding GAD-65 and a therapeutically effective amount of a second composition comprising a vector comprising a nucleic acid encoding GAD-67. In some embodiments, the first AAV vector and the second AAV vector are administered at the same time. In some embodiments, the first AAV vector is administered prior to the second AAV vector. In some embodiments, the second AAV vector is administered prior to the third AAV vector.

In embodiments, provided herein are methods of treating, preventing, and/or curing a neurodegenerative disease or disorder in a subject in need thereof, wherein the neurodegenerative disease or disorder is mediated by a GABA deficiency. In one aspect, the disclosure provides methods for treating diseases or disorders of the central nervous system associated with dopaminergic hypo activity, disease, injury or chemical lesioning. In one embodiment, the neurodegenerative disease or disorder is cognitive impairment. In one embodiment, the neurodegenerative disease or disorder is PD.

Vectors

In one aspect, provided is a method for the treatment of PD in a subject in need thereof, the method comprising administering to the subject one or more vectors comprising a nucleic acid sequence encoding a GAD. As used herein, a vector is a vehicle for delivering genetic material into a cell. In embodiments, the vector is a nucleic acid, including, but not limited to a plasmid, an episome, a RNA molecule, or a DNA molecule. In embodiments, the nucleic acid is circular. In embodiments, the nucleic acid is linear. In embodiments, the vector is a viral vector.

Vectors useful for the methods and compositions disclosed herein include vectors that are capable of autonomous replication (episomal vector) and/or vectors designed for gene expression in cells (expression vectors). In certain embodiments, the vectors described herein are expression vectors. Expression vectors allow expression of a nucleic acid in the target cell. An expression vector may contain both prokaryotic sequence to allow the propagation of the vector in bacteria and eukaryotic sequences to facilitate the expression of the encoded polypeptide in eukaryotic cells. The various methods employed in the preparation of plasmids and transformation of host organisms are known in the art.

In some embodiments, the GAD expression vector can be delivered via ex vivo gene therapy replacing lost cells with transplanted cells expressing GAD. An advantage of using such cells is the possibility of decreased immunoresistance as a patient's own cells can be used in an autotransplantation procedure.

In some embodiments, the vector can be delivered using a non-viral delivery system, for example. using a colloidal dispersion system such as macromolecule complexes, nanocapsules, microspheres, beads, and lipid-based systems including oil-in-water emulsions, micelles, mixed micelles, and liposomes.

In certain embodiments, the vectors described herein are viral vectors. Examples of viral vectors include, but are not limited to, retrovirus, adenovirus, parvovirus (e.g., adeno associated viruses, AAV), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses may include: avian leukosis-sarcoma, mammalian C-type, B-type viruses, D type viruses, HTLV-BLV group, lentivirus, and spumavirus.

In embodiments, the vectors comprising a nucleic acid encoding GAD are viral vectors used in gene therapy. The GAD transgene may be incorporated into any type of viral vectors that are used in gene therapy, such as recombinant retroviruses, adenovirus, adeno-associated virus (AAV), and herpes simplex virus-1.

In an embodiment, recombinant AAV (rAAV) is the vector(s) used for GAD transgene delivery. AAV particles comprise a linear, single-stranded AAV nucleic acid genome associated with an AAV capsid protein coat. AAV is incapable of replication without a helper virus which can be an adenovirus, vaccinia or herpes virus. In absence of a helper virus, AAV inserts its genome in the host cell chromosome assuming a latent state. Subsequent infection by the helper virus rescues the latent integrated copy which then replicates to produce infectious viral progeny.

Recombinant AAV (rAAV) vectors comprise a recombinant viral genome and capsid proteins. The rAAV genome comprising the GAD transgene(s) can be assembled from polynucleotides encoding the transgene(s), suitable regulatory elements, and viral elements necessary for packaging the rAAV genome. General methods for construction of rAAV genomes are known in the art. The AAV based expression vector may be composed of the AAV inverted terminal repeats (ITRs) flanking a restriction site that can be used for subcloning of the transgene, either directly using the restriction site available, or by excision of the transgene with restriction enzymes followed by polishing the ends and ligation into the AAV expression vector, optionally using linkers. A GAD transgene may be integrated in the AAV based expression vector along with one or more expression control elements including, for example an enhancer, promoter, and/or a post transcriptional regulatory sequence (PRE), flanked by AAV ITRs.

Methods for making rAAV vectors having a specific capsid protein are known in the art. Viral particles can be made by providing the components required for packaging the rAAV genome in a capsid in trans, or required components may be provided by an engineered host cell. Both of the methods use standard molecular biology techniques known to persons skilled in the art. Some or all of the required elements can be either under the control of an inducible or a constitutive promoter. The recombinant AAV genome, rep sequences, cap sequences, and helper functions for producing the rAAV may be delivered to the packaging host cell using any appropriate genetic element (vector). Typically, the recombinant AAVs are produced by transfecting a host cell with a recombinant AAV genome (comprising a transgene) to be packaged into AAV particles, an AAV helper function vector, and an accessory function vector. An AAV helper function vector encodes the AAV helper function sequences (i.e., rep and cap), which function in trans for productive AAV replication and encapsidation. The accessory function vector typically encodes the nucleotide sequences for non-AAV derived viral and/or cellular functions that are required for AAV replication including, without limitation, those elements involved in activation of AAV gene transcription, stage specific AAV mRNA splicing, AAV DNA replication, synthesis of cap expression products, and AAV capsid assembly.

As used herein, the terms “AAV1,” “AAV2,” “AAV3,” “AAV4,” and the like refer to AAV vectors containing inverted terminal repeats (ITR) from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

The AAV vectors described herein generally comprise a rAAV genome encoding one or more GAD transgenes operably linked to one or more regulatory elements in a manner that permits transgene transcription, translation, and/or expression in a target cell or a target tissue and is flanked by 5′ and 3′ ITRs. ITR sequences are typically about 145 bp in length. The AAV ITR sequences can be modified, e.g., by the insertion, deletion or substitution of one or more nucleotides by using standard molecular biology techniques provided the modification of the ITR sequence does not interfere with AAV vector function (such as efficient encapsidation of the rAAV genome. The AAV ITRs may be derived from any of the several AAV serotypes. The AAV ITR sequences at 3′ and 5′ can be identical or derived from different AAV serotype.

The expression control elements or regulatory elements operably linked to the transgene may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). In some embodiments, the transgene is a nucleic acid sequence, heterologous to the vector sequences, which encodes GAD-65 or GAD-67. Components of exemplary AAV vectors that may be used in conjunction with the compositions and methods of the disclosure are described herein.

Any appropriate AAV serotype or combination of AAV serotypes can be used in the methods and compositions of the present disclosure. Because the methods and compositions of the present disclosure are for the treatment and cure of neurodegenerative diseases or disorders, AAV serotypes that target at least the central nervous system can be used in some embodiments and include but are not limited to AAV1, AAV2, AAV4, AAV5, AAV6, AAV7, AAV8, AAV9, and AAV10.

Additionally, the rAAV genome includes expression control elements that are operably linked to the GAD transgene(s) in a manner which permits its transcription, translation and/or expression in a cell infected with the virus. As used herein, “operably linked” refers to a relationship between two or more nucleic acid sequences where certain nucleic acid sequences (e.g., control elements) influence characteristics of another nucleotide sequence (e.g., influencing expression of a transgene). Operably linked sequences include both expression control elements that are included in or are contiguous with the GAD transgene, and expression control elements that act in trans or at a distance to control expression of the transgene. Expression control elements as used herein include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. For example, as used herein, a nucleic acid sequence (e.g., a GAD coding sequence) and regulatory sequences are considered to be operably linked when they are covalently linked in such a way as to place the expression or transcription of the nucleic acid sequence under the influence or control of the regulatory sequences. In an embodiment, the human GAD-65 and/or GAD-67 are under regulation of the cytomegalovirus enhancer—chicken β-actin promoter and a woodchuck post-transcriptional regulatory element.

The promoter operably liked to a GAD transgene can either be inducible or constitutive. Inducible promoters allow regulation of gene expression and can be regulated by exogenously circumstances or compounds. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system; a ecdysone insect, a tetracycline-repressible system. Constitutive promoters are unregulated promoter that allows for continual transcription of its associated gene. Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter and a β-actin promoter.

In embodiments, a native promoter or fragment thereof for the GAD transgene may be used if the expression of the transgene to mimic the native expression is preferred. In another embodiment, a tissue specific promoter is used to allow expression in specific tissues for targeted gene therapy. Tissue specific promoters such as neuron-specific and glial-specific promoters allow the protein to express in the specific tissue desired. In an embodiment, the promoter is tissue specific and is essentially active only within the central nervous system or has a higher activity within the central nervous system. In an embodiment, the promoter can be specific for a particular cell type or neurons. In another embodiment, the promoter is specific for cells located in a particular region of the brain, for example, the cortex, subthalamic nucleus, stranium, nigra and/or hippocampus.

Some suitable neuronal specific promoters include, but are not limited to, neuron specific enolase (NSE) (GenBank Accession No: X51956), and human neurofilament light chain promoter (NEFL) (GenBank Accession No: L04147). Glial specific promoters include, but are not limited to, glial fibrillary acidic protein (GFAP) promoter (GenBank Accession No:M65210), S100 promoter (GenBank Accession No: M65210) and glutamine synthase promoter (GenBank Accession No: X59834).

In some embodiments, the viral vector is an rAAV vector, such as rAAV2-retro, AAV10, AAV2/10, AAV9 or an AAV2/9. In some embodiments, the composition comprising nucleic acids encoding GAD-65 and/or GAD-67 is administered in patients with advanced PD. In some embodiments, the administered composition comprises the nucleic acids encoding GAD-65 and GAD-67. In some embodiments, the method comprises administering one or more compositions comprising vectors comprising a nucleic acid encoding GAD-65 and a nucleic acid encoding GAD-67; either simultaneously or sequentially. In some embodiments, the method comprises administering one or more compositions comprising a nucleic acid encoding GAD-65, a nucleic acid encoding GAD-67, and other medications used to treat PD; either simultaneously or sequentially.

An effective amount of a rAAV is an amount sufficient to infect a sufficient number of cells of a target tissue in a subject to which the rAAV is administered. An effective amount of rAAV may be defined as an amount sufficient to have a therapeutic benefit on the subject, for example to improve in the subject one or more symptoms of the disease. The effective amount may vary depending on the species, age, weight, health of the subject and the CNS tissue to be targeted. Depending on the mode of administration the effective amount may vary as well. In some cases multiple doses of rAAV are administered to achieve the effective amount for the therapeutic benefit intended. The effective amount may vary depending on the serotype of rAAV. In certain cases, the effective amount of rAAV is 10¹⁰, 10¹¹, 10¹², 10¹³, 10¹⁴ or 10¹⁵ genome copies per subject. In some embodiments, the concentration of the AAV vector/vectors comprising the GAD transgene(s) administered is about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², or about 1×10¹³ vector genome/ml.

Pharmaceutical Compositions

In one embodiment, the disclosure provides a pharmaceutical composition comprising one or more vectors comprising a nucleic acid encoding GAD-65 and one or more vectors comprising a nucleic acid encoding GAD-67. These compositions may be administered alone or in combination with other agents for the treatment of subjects suffering from PD. A pharmaceutical composition may refer to a composition comprising the vector or vectors and a pharmaceutically acceptable carrier and optionally, other materials, e.g., one or more inert components (for example, a detectable agent or label) or one or more active components. Pharmaceutically acceptable carriers may include one or more solvents, dispersion media, coatings, antibacterial and antifungal agents, isotonic and absorption delaying agents, and the like that are physiologically compatible. Compositions can include components such as adjuvants, diluents, binders, stabilizers, buffers, salts, lipophilic solvents, preservatives, or mixtures thereof. Examples of pharmaceutically acceptable carriers include but are not limited to water, saline, phosphate buffered saline, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers). Carbohydrates such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol may also be used as excipients.

Carriers may also encompass a buffer or pH adjusting agent such as a salt prepared from an organic acid or base. Examples of buffers include but are not limited to organic acid salts such as salts of citric acid, ascorbic acid, gluconic acid, carbonic acid, tartaric acid, succinic acid, acetic acid, or phthalic acid, Tris, tromethamine hydrochloride, and phosphate buffers. Additional carriers may include polymeric excipients or additives such as polyvinylpyrrolidones, ficolls (a polymeric sugar), dextrates (e.g., cyclodextrins, such as 2-hydroxypropyl-.quadrature-cyclodextrin), polyethylene glycols, flavoring agents, antimicrobial agents, sweeteners, antioxidants, antistatic agents, surfactants (e.g., polysorbates such as TWEEN 20® and TWEEN 80®), lipids (e.g., phospholipids, fatty acids), steroids (e.g., cholesterol), and chelating agents (e.g., EDTA).

### Methods of Administration

A method of delivering one or more GAD transgenes to a subject may comprise administering rAAV by a single or multiple routes of administration. For example, the rAAV may be administered to the subject by intravenous injection of an effective amount of rAAV that crosses the blood-brain barrier. Intrathecal administration or intracerebral administration, for example, by intraventricular injection may also be used to deliver an effective amount of rAAV to the CNS. In a non-limiting example, an effective amount of rAAV may be coadministered by two different routes of administration, for example, by intrathecal administration and by intracerebral administration. Co-administration can be performed at approximately the same time, or at different times.

The term “intrathecal administration” refers to the administration of an agent into the spinal canal, or into the subarachnoid space so that it reaches the cerebrospinal fluid (CSF). The term “intracerebral administration” refers to the administration of an agent in and/or around the brain. Intracerebral administration includes, but is not limited to, administration of an agent in the brain, pons, cerebellum, intracranial cavity, and meninges surrounding the brain. Intracerebral administration may include administration in the dura mater, arachnoid material and pia mater of the brain. Intracerebral administration may include, in some cases, administration of an agent in the cerebrospinal fluid (CSF) of the subarachnoid space surrounding the brain. Intracerebral administration may include, in some cases, the administration of an agent in the ventricles of the brain, for example, the right lateral ventricle, the left lateral ventricle, the third ventricle, the fourth ventricle.

Intracerebral injections may involve direct injections into and/or around the brain. In some cases, intracerebral administration involves injection using stereotactic procedures for precise delivery. Stereotactic microinjection techniques are well known in the art and have been used in the art for precise delivery of a vector to a specific part of the brain. The procedure involves the use of a computer and a three-dimensional scanning device. In this method, the subject being treated can be placed within a stereotactic frame base and then imaged using high resolution MRI to determine the three-dimensional positioning of the particular region to be treated. The MRI images can then be used to determine a precise target site for the microinjection of the composition. The stereotactic computer software provides three-dimensional coordinates that are precisely registered for the stereotactic frame. For intracranial delivery, bun holes are drilled above the entry site, and the stereotactic apparatus is used to position the needle and ensure implantation at a predetermined depth. For bilateral delivery, a cooling period may be implemented where a document manager manually confirms the target site with the surgeon to avoid any mistakes. The stereotactic microinjection can be used to deliver the vector to any specific part of the brain including but not limited to hippocampus, cortex, subthalamic nucleus and/or nigra. In some instances, a microinjection pump may be used deliver a composition of rAAV as described herein. The infusion rate may vary between 1 μl/minute to 100 μl/minute depending on various factors such as age of the subject, weight/size of the subject, serotype of the AAV, intracerebral region chosen etc. In another embodiment, the vector is delivered using other delivery methods suitable for localized delivery, such as localized permeation of the blood-brain barrier.

The dosage regimens required for the therapy may be adjusted to provide the optimum desired response (e.g., a therapeutic or prophylactic response). For example, a single dose may be administered, several divided doses may be administered over time or the dose may be proportionally reduced or increased depending on the subject's responsiveness to the therapy.

The vectors described herein may be used in conjunction with one or more therapeutic agents that are not GAD. For example, the therapy may be used in combination to dopamine replacement therapies such as levodopa or carbidopa, dopamine agonists such as pramipexole, ropinerole, and bromocriptine, MAO-inhibitors such as selegline and rasagilene, Catechol O-methyltransferase (COMT) inhibitors such as entracapone and tolcapone, and various other compounds including, without limitation, any agent known in the art to treat one or more symptoms associated with PD as described herein. The vector or vectors may be administered to the subject suffering from PD simultaneously with other agents being used to treat the disease or separately.

Kits as described herein can include any combination of agents, compositions, components, reagents, administration devices or mechanisms, or other entities provided herein. For instance, a kit as described herein may include one or more AAV-GAD vectors and one or more of a carrier composition, an administration device, and a combination therapy agent. Kits may further include a device to facilitate delivery such as syringe for injection or a tool that facilitates the delivery of therapeutic compositions to the brain, e.g., the substantia nigra. Any of the kits provided herein can be included in a container, pack, or dispenser together with instructions for administration.

### EXAMPLES

### Example 1: Study Design

Identification of PD patient population most receptive to the disclosed method of treatment.

Patient Selection

Sixty-six patients with advanced PD were screened for eligibility to participate in a randomized, double-blind, sham surgery—controlled multicenter phase 2 trial of STN AAV2-GAD gene therapy and forty-five were randomized. All patients had progressive, levodopa-responsive PD as defined by the UK Parkinson's Disease Society criteria. Besides levodopa, other drugs for this disorder were allowed if no change in dose or drug type was made for 4 weeks or more before enrolment. An overnight off—medication unified Parkinson's disease rating scale (UPDRS) part 3 summed sub score (motor score) of 25 or more was required. Additional inclusion criteria were age 30-75 years, duration of symptoms of PD for at least 5 years, and levodopa responsiveness for at least 12 months. Patients could not have had previous brain surgery, used dopamine-receptor blocking drugs, had focal neurological deficits, or had abnormal cranial MRI; ¹⁸F-fluorodeoxyglucose PET scans needed to be compatible with PD, according to criteria for a metabolic brain pattern specific to PD that excluded patients with atypical Parkinsonism or indeterminate patterns. Patients were also excluded for cognitive impairment as defined by a Mattis dementia rating scale score of less than 130.

Randomization

A statistician and a programmer at PharmaNet Inc (each with no further role in the study) generated the randomization code. Before randomization, all subjects underwent metabolic brain imaging in the resting state with FDG PET. After screening to eliminate atypical parkinsonian conditions, 45 subjects with PD were randomized 1:1 to receive either STN AAV2-GAD gene therapy (n=22) or sham surgery (n=23). When patients arrived at the operating theatre, the neurosurgeon opened an envelope with the computer-generated random treatment assignment (ratio 1:1) for either AAV2-GAD or the sham procedure. Patients, caregivers, and investigators were masked to treatment assignment. For sham-assigned patients, the operating room team enacted a previously rehearsed plan for simulating a bilateral stereotaxic procedure identical to that done for the AAV2-GAD group. Those treated with sham received partial-thickness burr holes after a stereotaxic frame was placed. The simulation included sounds of microelectrode electrophysiological recording; infusion pumps and external catheters infusing normal saline into the burr hole site were used exactly as for patients receiving AAV2-GAD infusion. Masking was carefully planned for all information about treatment assignment, and no deviations occurred at any site. All raters were masked to treatment allocation and had no access to sequestered postoperative images and surgical records. On the third day after surgery and at all subsequent visits, patients were questioned for opinions about treatment assignments. Out of the forty-five initial subjects, 8 were excluded from the data analysis prior to unblinding due to failure of drug delivery (pump failure, inaccurate targeting of STN), based upon predetermined criteria.

Post-Treatment Selection of Patient Population for Analysis

The subjects and investigators were blinded to the treatment status for at least 6 months after the procedure; 6 subjects in the treatment groups and 2 subjects in the sham group were excluded from analysis because of missed surgical target or catheter/pump malfunctions. At baseline, there were no group differences in age, gender, UPDRS motor ratings, or cognitive tests (P>0.07). The subjects were rescanned under the blind 6 months after surgery (with the exception of one subject in each group) and again at the conclusion of the study at 12 months. The subjects were simultaneously unblinded after the final participant completed 6 months of blinded follow-up. The surgical procedures were staggered over a 1-year period, so the majority of participants [16 of 22 (73%) in the sham group; 11 of 20 (55%) in the GAD group] underwent imaging at 12 months after unblinding, while the remaining 6 sham and 9 GAD subjects were still under the blind at this 12-month time point.

### Example 2: Viral Vector Construction

AAV-GAD plasmids were generated that contained DNA encoding the open reading frame of human GAD-65 or GAD-67 under regulation of the cytomegalovirus enhancer—chicken β-actin promoter and woodchuck post-transcriptional regulatory element. Recombinant AAV genomes were packaged in human embryonic kidney (HEK) 293 cells and purified by heparin affinity chromatography, according to standard procedures and as previously described. The final formulation buffer was 1×phosphate-buffered saline solution. The genomic vector titers were measured by absolute quantification with the ABI7000 Sequence Detection System (Applied Biosystems, Foster City, Calif., USA).

### Example 3: Vector Delivery

The viruses encoding GAD-65 or GAD-67 were mixed in a 1-to-1 ratio and diluted to 1×10¹¹ viral genomes (vg)/mL (low dose), 3×10¹¹ vg/mL (medium dose), and 1×10¹² vg/mL (high dose) with 2×phosphate-buffered saline solution. The bulk harvest and final formulated products were rigorously examined with lot-release testing, as per FDA guidelines. Biosafety testing for mycoplasma, endotoxin, sterility, and adventitious viruses, and a general safety test were undertaken (AppTec Laboratory Services, Philadelphia, USA). Sham treatment was performed with saline solution.

The subthalamic nucleus was localized with the Leksell stereotactic frame and MRI image guidance. Standard intraoperative microelectrode recording was done with subjects awake to verify the precise location of the subthalamic nucleus. The tip of the microelectrode was then withdrawn to what was believed to be the center of the subthalamic nucleus. After microelectrode removal, a guide tube was inserted to 10 mm above the center of the nucleus. A catheter with a 10 mm tip of 200 μM I diameter was flushed with AAV2-GAD infusate and was inserted into the putative center of the nucleus. The catheter was locked in place with a cap containing a rip-cord tethering the catheter for post-procedure release at bedside. The scalp was closed after placement of first catheter to avoid catheter occlusion. A dose of AAV2-GAD per hemisphere (35 μl of 1×10¹² genomes/ml) was given bilaterally to the subthalamic nucleus of a PD subject. The procedure was performed on both sides of the scalp. The beginning of surgery on each side of the brain is preceded with a time-out when a study coordinator or other surgical team member confirmed with the coordinates noted by the surgeon and documented in writing before penetration of the brain. Another group of PD subjects received a dose of AAV2-GAD per hemisphere (35 μl of 1×10¹¹ genomes/ml) unilaterally to the subthalamic nucleus. Another group of PD subjects received a dose of AAV2-GAD per hemisphere (35 μl of 3×10¹¹ genomes/ml) unilaterally to the subthalamic nucleus. Another group of PD subjects received a dose of AAV2-GAD per hemisphere (35 μl of 1×10¹² genomes/ml) unilaterally to the subthalamic nucleus. Following completion of infusions, a fine-cut head CT scan was performed to determine the location of catheter tip locations. Post-catheter CT and MRI scans were performed on all subjects on 24 and 48 hour interval post-treatment.

### Example 3: Determination of “On-Time” Post Treatment

Subjects receiving either AAV-GAD (GAD) into the subthalamic nucleus or sham surgery (sham) recorded hourly ON and OFF times in a diary throughout each day for two weeks at each of the listed time periods (1, 3, 6 and 12 months after surgery). The average daily ON and OFF time over the two week period determined from the diaries were compared with baseline diaries taken within the 30 days prior to surgery in order to determine the change in the number of hours of ON time following surgery using two-tailed T test. At 12 months following surgery, the change in average daily ON time from baseline per subject in the AAV-GAD treatment group was correlated against the change in clinical scores for the same subjects across the same time period. The clinical score used was the part 3 of the UPDRS.

SEQUENCES

SEQ ID NO: 1
MASPGSGFWSFGSEDGSGDSENPGTARAWCQVAQKFTGGIGNKLCALLYGDAEKPAES
GGSQPPRAAARKAACACDQKPCSCSKVDVNYAFLHATDLLPACDGERPTLAFLQDVM
NILLQYVVKSFDRSTKVIDFHYPNELLQEYNWELADQPQNLEEILMHCQTTLKYAIKTG
HPRYFNQLSTGLDMVGLAADWLTSTANTNMFTYEIAPVFVLLEYVTLKKMREIIGWPG
GSGDGIFSPGGAISNMYAMMIARFKMFPEVKEKGMAALPRLIAFTSEHSHFSLKKGAAA
LGIGTDSVILIKCDERGKMIPSDLERRILEAKQKGFVPFLVSATAGTTVYGAFDPLLAVAD
ICKKYKIWMHVDAAWGGGLLMSRKHKWKLSGVERANSVTWNPHKMMGVPLQCSAL
LVREEGLMQNCNQMHASYLFQQDKHYDLSYDTGDKALQCGRHVDVFKLWLMWRAK
GTTGFEAHVDKCLELAEYLYNIIKNREGYEMVFDGKPQHTNVCFWYIPPSLRTLEDNEE
RMSRLSKVAPVIKARMMEYGTTMVSYQPLGDKVNFFRMVISNPAATHQDIDFLIEEIER
LGQDL
SEQ ID NO: 2
ATGGCATCTCCGGGCTCTGGCTTTTGGTCTTTCGGGTCGGAAGATGGCTCTGGGGAT
TCCGAGAATCCCGGCACAGCGCGAGCCTGGTGCCAAGTGGCTCAGAAGTTCACGGG
CGGCATCGGAAACAAACTGTGCGCCCTGCTCTACGGAGACGCCGAGAAGCCGGCGG
AGAGCGGCGGGAGCCAACCCCCGCGGGCCGCCGCCCGGAAGGCCGCCTGCGCCTGC
GACCAGAAGCCCTGCAGCTGCTCCAAAGTGGATGTCAACTACGCGTTTCTCCATGCA
ACAGACCTGCTGCCGGCGTGTGATGGAGAAAGGCCCACTTTGGCGTTTCTGCAAGAT
GTTATGAACATTTTACTTCAGTATGTGGTGAAAAGTTTCGATAGATCAACCAAAGTG
ATTGATTTCCATTATCCTAATGAGCTTCTCCAAGAATATAATTGGGAATTGGCAGAC
CAACCACAAAATTTGGAGGAAATTTTGATGCATTGCCAAACAACTCTAAAATATGCA
ATTAAAACAGGGCATCCTAGATACTTCAATCAACTTTCTACTGGTTTGGATATGGTT
GGATTAGCAGCAGACTGGCTGACATCAACAGCAAATACTAACATGTTCACCTATGA
AATTGCTCCAGTATTTGTGCTTTTGGAATATGTCACACTAAAGAAAATGAGAGAAAT
CATTGGCTGGCCAGGGGGCTCTGGCGATGGGATATTTTCTCCCGGTGGCGCCATATC
TAACATGTATGCCATGATGATCGCACGCTTTAAGATGTTCCCAGAAGTCAAGGAGAA
AGGAATGGCTGCTCTTCCCAGGCTCATTGCCTTCACGTCTGAACATAGTCATTTTTCT
CTCAAGAAGGGAGCTGCAGCCTTAGGGATTGGAACAGACAGCGTGATTCTGATTAA
ATGTGATGAGAGAGGGAAAATGATTCCATCTGATCTTGAAAGAAGGATTCTTGAAG
CCAAACAGAAAGGGTTTGTTCCTTTCCTCGTGAGTGCCACAGCTGGAACCACCGTGT
ACGGAGCATTTGACCCCCTCTTAGCTGTCGCTGACATTTGCAAAAAGTATAAGATCT
GGATGCATGTGGATGCAGCTTGGGGTGGGGGATTACTGATGTCCCGAAAACACAAG
TGGAAACTGAGTGGCGTGGAGAGGGCCAACTCTGTGACGTGGAATCCACACAAGAT
GATGGGAGTCCCTTTGCAGTGCTCTGCTCTCCTGGTTAGAGAAGAGGGATTGATGCA
GAATTGCAACCAAATGCATGCCTCCTACCTCTTTCAGCAAGATAAACATTATGACCT
GTCCTATGACACTGGAGACAAGGCCTTACAGTGCGGACGCCACGTTGATGTTTTTAA
ACTATGGCTGATGTGGAGGGCAAAGGGGACTACCGGGTTTGAAGCGCATGTTGATA
AATGTTTGGAGTTGGCAGAGTATTTATACAACATCATAAAAAACCGAGAAGGATAT
GAGATGGTGTTTGATGGGAAGCCTCAGCACACAAATGTCTGCTTCTGGTACATTCCT
CCAAGCTTGCGTACTCTGGAAGACAATGAAGAGAGAATGAGTCGCCTCTCGAAGGT
GGCTCCAGTGATTAAAGCCAGAATGATGGAGTATGGAACCACAATGGTCAGCTACC
AACCCTTGGGAGACAAGGTCAATTTCTTCCGCATGGTCATCTCAAACCCAGCGGCAA
CTCACCAAGACATTGACTTCCTGATTGAAGAAATAGAACGCCTTGGACAAGATTTAT
AA
SEQ ID NO: 3
MSPIHHHHHHLVPRGSEASNSGFWSFGSEDGSGDSENPGTARAWCQVAQKFTGGIGNK
LCALLYGDAEKPAESGGSQPPRAAARKAACACDQKPCSCSKVDVNYAFLHATDLLPAC
DGERPTLAFLQDVMNILLQYVVKSFDRSTKVIDFHYPNELLQEYNWELADQPQNLEEIL
MHCQTTLKYAIKTGHPRYFNQLSTGLDMVGLAADWLTSTANTNMFTYEIAPVFVLLEY
VTLKKMREIIGWPGGSGDGIFSPGGAISNMYAMMIARFKMFPEVKEKGMAALPRLIAFT
SEHSHFSLKKGAAALGIGTDSVILIKCDERGKMIPSDLERRILEAKQKGFVPFLVSATAGT
TVYGAFDPLLAVADICKKYKIWMHVDAAWGGGLLMSRKHKWKLSGVERANSVTWNP
HKMMGVPLQCSALLVREEGLMQNCNQMHASYLFQQDKHYDLSYDTGDKALQCGRHV
DVFKLWLMWRAKGTTGFEAHVDKCLELAEYLYNIIKNREGYEMVFDGKPQHTNVCFW
YIPPSLRTLEDNEERMSRLSKVAPVIKARMMEYGTTMVSYQPLGDKVNFFRMVISNPAA
THQDIDFLIEEIERLGQDL
SEQ ID NO: 4
ATGTCCCCTATACATCACCATCACCATCACCTGGTTCCGCGTGGATCCGAAGCTTCG
AATTCTGGCTTTTGGTCTTTCGGGTCGGAAGATGGCTCTGGGGATTCCGAGAATCCC
GGCACAGCGCGAGCCTGGTGCCAAGTGGCTCAGAAGTTCACGGGCGGCATCGGAAA
CAAACTGTGCGCCCTGCTCTACGGAGACGCCGAGAAGCCGGCGGAGAGCGGCGGGA
GCCAACCCCCGCGGGCCGCCGCCCGGAAGGCCGCCTGCGCCTGCGACCAGAAGCCC
TGCAGCTGCTCCAAAGTGGATGTCAACTACGCGTTTCTCCATGCAACAGACCTGCTG
CCGGCGTGTGATGGAGAAAGGCCCACTTTGGCGTTTCTGCAAGATGTTATGAACATT
TTACTTCAGTATGTGGTGAAAAGTTTCGATAGATCAACCAAAGTGATTGATTTCCAT
TATCCTAATGAGCTTCTCCAAGAATATAATTGGGAATTGGCAGACCAACCACAAAAT
TTGGAGGAAATTTTGATGCATTGCCAAACAACTCTAAAATATGCAATTAAAACAGG
GCATCCTAGATACTTCAATCAACTTTCTACTGGTTTGGATATGGTTGGATTAGCAGC
AGACTGGCTGACATCAACAGCAAATACTAACATGTTCACCTATGAAATTGCTCCAGT
ATTTGTGCTTTTGGAATATGTCACACTAAAGAAAATGAGAGAAATCATTGGCTGGCC
AGGGGGCTCTGGCGATGGGATATTTTCTCCCGGTGGCGCCATATCTAACATGTATGC
CATGATGATCGCACGCTTTAAGATGTTCCCAGAAGTCAAGGAGAAAGGAATGGCTG
CTCTTCCCAGGCTCATTGCCTTCACGTCTGAACATAGTCATTTTTCTCTCAAGAAGGG
AGCTGCAGCCTTAGGGATTGGAACAGACAGCGTGATTCTGATTAAATGTGATGAGA
GAGGGAAAATGATTCCATCTGATCTTGAAAGAAGGATTCTTGAAGCCAAACAGAAA
GGGTTTGTTCCTTTCCTCGTGAGTGCCACAGCTGGAACCACCGTGTACGGAGCATTT
GACCCCCTCTTAGCTGTCGCTGACATTTGCAAAAAGTATAAGATCTGGATGCATGTG
GATGCAGCTTGGGGTGGGGGATTACTGATGTCCCGAAAACACAAGTGGAAACTGAG
TGGCGTGGAGAGGGCCAACTCTGTGACGTGGAATCCACACAAGATGATGGGAGTCC
CTTTGCAGTGCTCTGCTCTCCTGGTTAGAGAAGAGGGATTGATGCAGAATTGCAACC
AAATGCATGCCTCCTACCTCTTTCAGCAAGATAAACATTATGACCTGTCCTATGACA
CTGGAGACAAGGCCTTACAGTGCGGACGCCACGTTGATGTTTTTAAACTATGGCTGA
TGTGGAGGGCAAAGGGGACTACCGGGTTTGAAGCGCATGTTGATAAATGTTTGGAG
TTGGCAGAGTATTTATACAACATCATAAAAAACCGAGAAGGATATGAGATGGTGTT
TGATGGGAAGCCTCAGCACACAAATGTCTGCTTCTGGTACATTCCTCCAAGCTTGCG
TACTCTGGAAGACAATGAAGAGAGAATGAGTCGCCTCTCGAAGGTGGCTCCAGTGA
TTAAAGCCAGAATGATGGAGTATGGAACCACAATGGTCAGCTACCAACCCTTGGGA
GACAAGGTCAATTTCTTCCGCATGGTCATCTCAAACCCAGCGGCAACTCACCAAGAC
ATTGACTTCCTGATTGAAGAAATAGAACGCCTTGGACAAGATTTATAA
SEQ ID NO: 5
MASSTPSSSATSSNAGADPNTTNLRPTTYDTWCGVAHGCTRKLGLKICGFLQRTNSLEE
KSRLVSAFRERQSSKNLLSCENSDRDARFRRTETDFSNLFARDLLPAKNGEEQTVQFLLE
VVDILLNYVRKTFDRSTKVLDFHHPHQLLEGMEGFNLELSDHPESLEQILVDCRDTLKY
GVRTGHPRFFNQLSTGLDIIGLAGEWLTSTANTNMFTYEIAPVFVLMEQITLKKMREIVG
WSSKDGDGIFSPGGAISNMYSIMAARYKYFPEVKTKGMAAVPKLVLFTSEQSHYSIKKA
GAALGFGTDNVILIKCNERGKIIPADFEAKILEAKQKGYVPFYVNATAGTTVYGAFDPIQ
EIADICEKYNLWLHVDAAWGGGLLMSRKHRHKLNGIERANSVTWNPHKMMGVLLQCS
AILVKEKGILQGCNQMCAGYLFQPDKQYDVSYDTGDKAIQCGRHVDIFKFWLMWKAK
GTVGFENQINKCLELAEYLYAKIKNREEFEMVFNGEPEHTNVCFWYIPQSLRGVPDSPQ
RREKLHKVAPKIKALMMESGTTMVGYQPQGDKANFFRMVISNPAATQSDIDFLIEEIER
LGQDL
SEQ ID NO: 6:
ATGGCGTCTTCGACCCCATCTTCGTCCGCAACCTCCTCGAACGCGGGAGCGGACCCC
AATACCACTAACCTGCGCCCCACAACGTACGATACCTGGTGCGGCGTGGCCCATGG
ATGCACCAGAAAACTGGGGCTCAAGATCTGCGGCTTCTTGCAAAGGACCAACAGCC
TGGAAGAGAAGAGTCGCCTTGTGAGTGCCTTCAGGGAGAGGCAATCCTCCAAGAAC
CTGCTTTCCTGTGAAAACAGCGACCGGGATGCCCGCTTCCGGCGCACAGAGACTGA
CTTCTCTAATCTGTTTGCTAGAGATCTGCTTCCGGCTAAGAACGGTGAGGAGCAAAC
CGTGCAATTCCTCCTGGAAGTGGTGGACATACTCCTCAACTATGTCCGCAAGACATT
TGATCGCTCCACCAAGGTGCTGGACTTTCATCACCCACACCAGTTGCTGGAAGGCAT
GGAGGGCTTCAACTTGGAGCTCTCTGACCACCCCGAGTCCCTGGAGCAGATCCTGGT
TGACTGCAGAGACACCTTGAAGTATGGGGTTCGCACAGGTCATCCTCGATTTTTCAA
CCAGCTCTCCACTGGATTGGATATTATTGGCCTAGCTGGAGAATGGCTGACATCAAC
GGCCAATACCAACATGTTTACATATGAAATTGCACCAGTGTTTGTCCTCATGGAACA
AATAACACTTAAGAAGATGAGAGAGATAGTTGGATGGTCAAGTAAAGATGGTGATG
GGATATTTTCTCCTGGGGGCGCCATATCCAACATGTACAGCATCATGGCTGCTCGCT
ACAAGTACTTCCCGGAAGTTAAGACAAAGGGCATGGCGGCTGTGCCTAAACTGGTC
CTCTTCACCTCAGAACAGAGTCACTATTCCATAAAGAAAGCTGGGGCTGCACTTGGC
TTTGGAACTGACAATGTGATTTTGATAAAGTGCAATGAAAGGGGGAAAATAATTCC
AGCTGATTTTGAGGCAAAAATTCTTGAAGCCAAACAGAAGGGATATGTTCCCTTTTA
TGTCAATGCAACTGCTGGCACGACTGTTTATGGAGCTTTTGATCCGATACAAGAGAT
TGCAGATATATGTGAGAAATATAACCTTTGGTTGCATGTCGATGCTGCCTGGGGAGG
TGGGCTGCTCATGTCCAGGAAGCACCGCCATAAACTCAACGGCATAGAAAGGGCCA
ACTCAGTCACCTGGAACCCTCACAAGATGATGGGCGTGCTGTTGCAGTGCTCTGCCA
TTCTCGTCAAGGAAAAGGGTATACTCCAAGGATGCAACCAGATGTGTGCAGGATAT
CTCTTCCAGCCAGACAAGCAGTATGATGTCTCCTACGACACCGGGGACAAGGCAAT
TCAGTGTGGCCGCCACGTGGATATCTTCAAGTTCTGGCTGATGTGGAAAGCAAAGGG
CACAGTGGGATTTGAAAACCAGATCAACAAATGCCTGGAACTGGCTGAATACCTCT
ATGCCAAGATTAAAAACAGAGAAGAATTTGAGATGGTTTTCAATGGCGAGCCTGAG
CACACAAACGTCTGTTTTTGGTATATTCCACAAAGCCTCAGGGGTGTGCCAGACAGC
CCTCAACGACGGGAAAAGCTACACAAGGTGGCTCCAAAAATCAAAGCCCTGATGAT
GGAGTCAGGTACGACCATGGTTGGCTACCAGCCCCAAGGGGACAAGGCCAACTTCT
TCCGGATGGTCATCTCCAACCCAGCCGCTACCCAGTCTGACATTGACTTCCTCATTG
AGGAGATAGAAAGACTGGGCCAGGATCTGTAA
SEQ ID NO: 7
MASSTPSSSATSSNAGADPNTTNLRPTTYDTWCGVAHGCTRKLGLKICGFLQRTNSLEE
KSRLVSAFKERQSSKNLLSCENSDRDARFRRTETDFSNLFARDLLPAKNGEEQTVQFLLE
VVDILLNYVRKTFDRSTKVLDFHHPHQLLEGMEGFNLELSDHPESLEQILVDCRDTLKY
GVRTGHPRFFNQLSTGLDIIGLAGEWLTSTANTNMFTYEIAPVFVLMEQITLKKMREIVG
WSSKDGDGIFSPGGAISNMYSIMAARYKYFPEVKTKGMAAVPKLVLFTSEQSHYSIKKA
GAALGFGTDNVILIKCNERGKIIPADFEAKILEAKQKGYVPFYVNATAGTTVYGAFDPIQ
EIADICEKYNLWLHVDAAWGGGLLMSRKHRHKLNGIERANSVTWNPHKMMGVLLQCS
AILVKEKGILQGCNQMCAGYLFQPDKQYDVSYDTGDKAIQCGRHVDIFKFWLMWKAK
GTVGFENQINKCLELAEYLYAKIKNREEFEMVFNGEPEHTNVCFWYIPQSLRGVPDSPQ
RREKLHKVAPKIKALMMESGTTMVGYQPQGDKANFFRMVISNPAATQSDIDFLIEEIER
LGQDL
SEQ ID NO: 8
ATGGCGTCTTCGACCCCATCTTCGTCCGCAACCTCCTCGAACGCGGGAGCGGACCCC
AATACCACTAACCTGCGCCCCACAACGTACGATACCTGGTGCGGCGTGGCCCATGG
ATCACCAGAAAACTGGGGCTCAAGATCTGCGGCTTCTTGCAAAGGACCAACAGCC
TGGAAGAGAAGAGTCGCCTTGTGAGTGCCTTCAAGGAGAGGCAATCCTCCAAGAAC
CTGCTTTCCTGTGAAAACAGCGACCGGGATGCCCGCTTCCGGCGCACAGAGACTGA
CTTCTCTAATCTGTTTGCTAGAGATCTGCTTCCGGCTAAGAACGGTGAGGAGCAAAC
CGTGCAATTCCTCCTGGAAGTGGTGGACATACTCCTCAACTATGTCCGCAAGACATT
TGATCGCTCCACCAAGGTGCTGGACTTTCATCACCCACACCAGTTGCTGGAAGGCAT
GGAGGGCTTCAACTTGGAGCTCTCTGACCACCCCGAGTCCCTGGAGCAGATCCTGGT
TGACTGCAGAGACACCTTGAAGTATGGGGTTCGCACAGGTCATCCTCGATTTTTCAA
CCAGCTCTCCACTGGATTGGATATTATTGGCCTAGCTGGAGAATGGCTGACATCAAC
GGCCAATACCAACATGTTTACATATGAAATTGCACCAGTGTTTGTCCTCATGGAACA
AATAACACTTAAGAAGATGAGAGAGATAGTTGGATGGTCAAGTAAAGATGGTGATG
GGATATTTTCTCCTGGGGGCGCCATATCCAACATGTACAGCATCATGGCTGCTCGCT
ACAAGTACTTCCCGGAAGTTAAGACAAAGGGCATGGCGGCTGTGCCTAAACTGGTC
CTCTTCACCTCAGAACAGAGTCACTATTCCATAAAGAAAGCTGGGGCTGCACTTGGC
TTTGGAACTGACAATGTGATTTTGATAAAGTGCAATGAAAGGGGGAAAATAATTCC
AGCTGATTTTGAGGCAAAAATTCTTGAAGCCAAACAGAAGGGATATGTTCCCTTTTA
TGTCAATGCAACTGCTGGCACGACTGTTTATGGAGCTTTTGATCCGATACAAGAGAT
TGCAGATATATGTGAGAAATATAACCTTTGGTTGCATGTCGATGCTGCCTGGGGAGG
TGGGCTGCTCATGTCCAGGAAGCACCGCCATAAACTCAACGGCATAGAAAGGGCCA
ACTCAGTCACCTGGAACCCTCACAAGATGATGGGCGTGCTGTTGCAGTGCTCTGCCA
TTCTCGTCAAGGAAAAGGGTATACTCCAAGGATGCAACCAGATGTGTGCAGGATAC
CTCTTCCAGCCAGACAAGCAGTATGATGTCTCCTACGACACCGGGGACAAGGCAAT
TCAGTGTGGCCGCCACGTGGATATCTTCAAGTTCTGGCTGATGTGGAAAGCAAAGGG
CACAGTGGGATTTGAAAACCAGATCAACAAATGCCTGGAACTGGCTGAATACCTCT
ATGCCAAGATTAAAAACAGAGAAGAATTTGAGATGGTTTTCAATGGCGAGCCTGAG
CACACAAACGTCTGTTTTTGGTATATTCCACAAAGCCTCAGGGGTGTGCCAGACAGC
CCTCAACGACGGGAAAAGCTACACAAGGTGGCTCCAAAAATCAAAGCCCTGATGAT
GGAGTCAGGTACGACCATGGTTGGCTACCAGCCCCAAGGGGACAAGGCCAACTTCT
TCCGGATGGTCATCTCCAACCCAGCCGCTACCCAGTCTGACATTGACTTCCTCATTG
AGGAGATAGAAAGACTGGGCCAGGATCTGTAA
SEQ ID NO: 9
MASSTPSSSATSSNAGADPNTTNLRPTTYDTWCGVAHGCTRKLGLKICGFLQRTNSLEE
KSRLVSAFKERQSSKNLLSCENSDRDARFRRTETDFSNLFARDLLPAKNGEEQTVQFLLE
VVDILLNYVRKTFDRSTKVLDFHHPHQLLEGMEGFNLELSDHPESLEQILVDCRDTLKY
GVRTGHPRFFNQLSTGLDIIGLAGEWLTSTANTNMPSDMRECWLLR

We claim:

1. A method of treating Parkinson's disease (PD) in a subject in need thereof, the method comprising:
(a) identifying a subject having less than 10 hours of on-time per day; and
(b) administering to the subject a composition comprising a therapeutically effective amount of one or more vectors to the subthalamic nucleus of the patient, wherein each vector comprises a nucleic acid sequence encoding glutamic acid decarboxylase (GAD) and wherein the subject's on-time is increased.

2. The method of claim 1, where the subject has less than 8 hours of on-time per day before treatment.

3. The method of claim 1, wherein the subject scores 30 or more on part III of the UPDRS in the off medication state.

4. The method of claim 1, wherein the one or more vectors are introduced bilaterally to the subthalamic nucleus of the patient.

5. The method of claim 1, wherein the one or more vectors comprises a nucleic acid sequence encoding GAD-65 and a nucleic acid sequence encoding GAD-67.

6. The method of claim 5, wherein the composition comprises about a 1:1 ratio of vectors encoding GAD-65 and vectors encoding GAD-67.

7. The method of claim 1, where the one or more vectors are viral vectors.

8. The method of claim 7, wherein the viral vectors are adeno-associated virus (AAV) vectors.

9. The method of claim 8, wherein the composition comprises at least 1×10¹¹ vector genomes/ml.

10. The method of claim 8, where the composition comprises at least 3×10¹¹ vector genome/ml.

11. The method of claim 8, where the composition comprises at least 1×10¹² vector genome/ml.

12. The method of claim 1, wherein the subject shows an increase of at least 40% in on-time 12 months post treatment.

13. The method of claim 1, where the subject shows at least 30% increase in on-time after 12 months of post treatment.

14. The method of any claim 1, where the subject shows at least 20% increase in on-time after 12 months of post treatment.


SEQUENCE LISTING (9 sequences)

SEQ ID NO: 1 - protein, 585 aa, HOMO SAPIENS
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 2 - DNA, 1758 bp, HOMO SAPIENS
atggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataa

SEQ ID NO: 3 - protein, 600 aa, HOMO SAPIENS [sequence omitted]

SEQ ID NO: 4 - DNA, 1803 bp, HOMO SAPIENS
atgtcccctatacatcaccatcaccatcacctggttccgcgtggatccgaagcttcgaattctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataa

SEQ ID NO: 5 - protein, 594 aa, HOMO SAPIENS
MASSTPSSSATSSNAGADPNTTNLRPTTYDTWCGVAHGCTRKLGLKICGFLQRTNSLEEKSRLVSAFRERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 6 - DNA, 1785 bp, HOMO SAPIENS
atggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcagggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatatctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 7 - protein, 594 aa, HOMO SAPIENS
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 8 - DNA, 1785 bp, HOMO SAPIENS
atggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 9 - protein, 224 aa, HOMO SAPIENS
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMPSDMRECWLLR